(19) United States
(12) Patent Application Publication
(10) Pub. No.: US 2025/0372228 A1
(43) Pub. Date: Dec. 4, 2025

(54) METHOD AND SYSTEM FOR DETERMINING POPULATION PHARMACOKINETIC MODEL OF PROPOFOL AND DERIVATIVE THEREOF

(71) Applicant: Xizang Haisco Pharmaceutical Co., Ltd., Lhoka (CN)

(72) Inventors: Xu WANG, Chengdu City (CN); Qianqian JI, Chengdu City (CN); Kai CHEN, Chengdu City (CN); Xiao LIU, Chengdu City (CN); Pangke YAN, Chengdu City (CN); Nan WU, Chengdu City (CN)

(73) Assignee: Xizang Haisco Pharmaceutical Co., Ltd., Lhoka (CN)

(21) Appl. No.: 19/297,500

(22) Filed: Aug. 12, 2025

Related U.S. Application Data
(63) Continuation-in-part of application No. 18/018,427, filed on Jan. 27, 2023.

(30) Foreign Application Priority Data
Aug. 3, 2020 (CN) 202010766611.2

(51) Int. Cl. G16H 20/17; G16H 10/60; G16H 50/70; G16H 70/40

(52) U.S. Cl. CPC G16H 20/17; G16H 10/60; G16H 50/70; G16H 70/40

(57) ABSTRACT

Provided are a method and system for determining a population pharmacokinetic model of propofol and a derivative thereof. The method comprises determining a population pharmacokinetic model of a compound of formula (I) or propofol, wherein an equation of pharmacokinetic parameters in the population pharmacokinetic model of the compound of formula (I) comprises: CLᵢ=exp(4.20÷0.349·log(WT/63.9)−0.749·log(TP/72.4)+0.238·SITE+η_{CL,i}); an equation of pharmacokinetic parameters in the population pharmacokinetic model of propofol comprises: CLᵢ=exp(4.56÷η_{CL,i}).

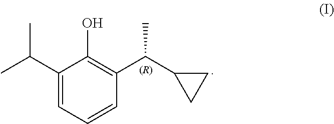

### CROSS-REFERENCE TO RELATED APPLICATIONS

This application is a continuation-in-part of U.S. application Ser. No. 18/018,427, filed Jan. 27, 2023, which is a U.S. 371 national application of International Application No. PCT/CN2021/109601, filed on Jul. 30, 2021, which claims priority to Chinese Patent Application No. 202010766611.2, filed on Aug. 3, 2020, each of which is hereby incorporated by reference in their entireties for any and all purposes.

### TECHNICAL FIELD

The present disclosure relates to the field of medicine, and specifically relates to a method and system for determining a population pharmacokinetic model of propofol and a derivative thereof.

### BACKGROUND ART

Propofol derivative injectable emulsion (hereinafter referred to as propofol derivative, with a chemical name of 2-[(1R)-1-cyclopropylethyl]-6-isopropyl-phenol), which is developed by Sichuan Haisco Pharmaceutical Co., Ltd. as a novel intravenous anesthetic with independent intellectual property rights, is intended to be used for sedation/anesthesia in various diagnostic examinations or treatments, induction and maintenance of general anesthesia, and sedation (ICU sedation) of intensive care subjects under mechanical ventilation. The propofol derivative, as the active ingredient, is a chemical entity similar to propofol, and is a single diastereomer with two chiral centers of R-configuration. The strategy of the medicinal chemistry design is to systematically improve the pharmacological and physicochemical properties of the drug for binding to a receptor to obtain a compound superior to propofol, i.e., the propofol derivative. The main action mechanism of the propofol derivative is to allow for the inflow of chloride ions by enhancing an ion channel mediated by a γ-aminobutyric acid type A (GABAA) receptor, thereby achieving the inhibition of the nervous centralis. The channel is also a main target where propofol exerts its effects. The propofol derivative has the pharmacodynamic characteristics of fast onset of action and stable and rapid recovery. Moreover, the propofol derivative has higher target selectivity and in-vitro and in-vivo activities and a potency 4-5 times that of propofol, and shows a more stable hemodynamics in animal trials. In addition, under the same conditions and concentrations, it is measured by using “an ultrafiltration method” that the propofol derivative has a lower free drug concentration in the aqueous phase as compared with propofol (Jingan®), suggesting that pain at an injection site may be reduced or eliminated.

### SUMMARY OF THE DISCLOSURE

An objective of the present disclosure is to provide a method for determining a population pharmacokinetic model of a propofol derivative.

An objective of the present disclosure is to quantitatively evaluate the influence of both intrinsic and extrinsic factors on the PK by using the method of the present disclosure. According to the population pharmacokinetic (PopPK) model established in the present disclosure, the individual exposure can be estimated for exposure-response (E-R) analysis. E-R analysis is critical for understanding the drug safety and efficacy. Dosage is a direct indicator of drug exposure commonly used in clinical trials, but the drug concentration in serum/plasma is a more direct indicator of exposure to a target of drug action and thus is correlated with the clinical efficacy and safety.

Another objective of the present disclosure is to provide a system for determining clinical individual administration parameters of a compound of formula (I) or propofol.

In order to achieve the above-mentioned objectives, on one hand, the present disclosure provides a method for determining a population pharmacokinetic model of a compound of formula (I) (the propofol derivative). The method comprises determining the population pharmacokinetic model of the compound of formula (I):

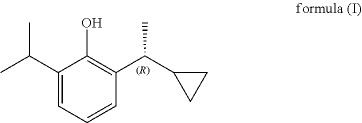

wherein an equation of pharmacokinetic parameters in the population pharmacokinetic model of the compound of formula (I) comprises:

$$\text{?} = exp ( 4.2 + 0.349 \cdot log ( WT / 63.9 ) - 0.749 \cdot log ( TP / 72.4 ) + 0.238 \cdot SITE + \text{?}$$

$$\text{?} \text{indicates text missing or illegible when filed}$$

wherein CLᵢ represents central compartment clearance of the ith subject; when collecting a sample from venous blood, SITE=0, and when collecting a sample from artery blood, SITE=1; WT represents weight; TP represents total protein; η_{CL,i} is interindividual variation of the CL of the ith subject; and η follows a normal distribution with mean 0 and variance ω2, wherein the ω2 is a diagonal element of a variance-covariance matrix (Ω) of the interindividual variation.

The present disclosure also provides a method for inducing and maintaining anesthesia, comprising administering the compound of formula (I) or propofol to a subject, wherein, dosage regimen of the compound of formula (I) or propofol is derived from a population pharmacokinetic model, obtained through the following methods:

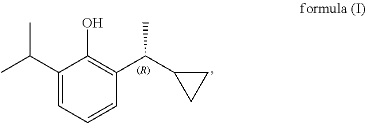

wherein an equation of pharmacokinetic parameters in the population pharmacokinetic model of the compound of formula (I) comprises:

$$\text{?} = exp ( 4.2 + 0.349 \cdot log ( WT / 63.9 ) - 0.749 \cdot log ( TP / 72.4 ) + 0.238 \cdot SITE + \text{?}$$

$$\text{?} \text{indicates text missing or illegible when filed}$$

The present disclosure provides a method for determining a population pharmacokinetic model of propofol. The method comprises determining the population pharmacokinetic model of propofol, wherein
an equation of pharmacokinetic parameters in the population pharmacokinetic model of propofol comprises:

$${CL}_{i} = exp ( 4.56 + \text{?} )$$

$$\text{?} \text{indicates text missing or illegible when filed}$$

wherein CLᵢ represents central compartment clearance of the ith subject; η_{CL,i} is interindividual variation of the CL of the ith subject; and η follows a normal distribution with mean 0 and variance ω2, wherein the ω2 is a diagonal element of a variance-covariance matrix (Ω) of the interindividual variation.

The above-mentioned model shows the relationship between the drug clearance and related covariates (weight, total protein and administration site). The influence of the covariates on the PK parameters (mainly the drug exposure) can be clinically evaluated by the model so as to guide the clinical administration. Firstly, the individual PK parameters of a subject are estimated by using a Bayesian post-hoc method on the basis of the PopPK model established in this study, and a plasma drug concentration-time curve of the propofol derivative or propofol administered by intravenous infusion is simulated according to the actual dosages of administration so as to calculate the area (the exposure) under the plasma drug concentration-time curve in different time periods. In addition, it is necessary to combine with the data analysis of correlation between the exposure and the drug efficacy and safety to give a reasonable dosage regimen.

The compound of formula (I) or propofol is administed to the subjects according to the dosage regimen.

The above-mentioned population pharmacokinetic models (PopPK models) of the present disclosure are ultimately selected from a three-compartment model with zero-order absorption and first-order linear elimination from the central compartment (as shown in FIG. 1). The PopPK model is composed of the following parameters: central compartment clearance (CL), volume of distribution in the central compartment (V1), volumes of distribution in the peripheral compartments (V2, V3), and intercompartmental clearances (Q2, Q3).

According to some specific embodiments of the present disclosure, the equation of the pharmacokinetic parameters in the population pharmacokinetic model of the compound of formula (I) further comprises:

$$\text{?} = exp ( 0.908 + 0.426 \cdot log ( AGE / 27 ) + \text{?} )$$

$$\text{?} = exp ( 4.06 + \text{?} )$$

$$\text{?} = exp ( 1.75 )$$

$$\text{?} = exp ( 4.08 + \text{?} )$$

$$\text{?} = exp ( 4.36 + \text{?} )$$

$$\text{?} \text{indicates text missing or illegible when filed}$$

wherein V₁ᵢ represents volume of distribution in the central compartment of the ith subject;
V₂ᵢ represents volume of distribution in the peripheral compartment 1 of the ith subject;
V₃ᵢ represents volume of distribution in the peripheral compartment 2 of the ith subject;
Q₂ᵢ represents intercompartmental clearance between the peripheral compartment 1 and the central compartment of the ith subject;
Q₃ᵢ represents intercompartmental clearance between the peripheral compartment 2 and the central compartment of the ith subject;
AGE represents age; and η represents interindividual variation of a corresponding parameter.

According to some specific embodiments of the present disclosure, the equation of the pharmacokinetic parameters in the population pharmacokinetic model of propofol further comprises:

$$\text{?} = exp ( 2.25 + \text{?} )$$

$$\text{?} = exp ( 4.96 )$$

$$\text{?} = exp ( 3.63 )$$

$$\text{?} = exp ( 3.88 )$$

$$\text{?} = exp ( 5.57 )$$

$$\text{?} \text{indicates text missing or illegible when filed}$$

According to some specific embodiments of the present disclosure, the method for determining the population pharmacokinetic model of the compound of formula (I) and propofol comprises obtaining the population pharmacokinetic model according to the influence of the covariates on the pharmacokinetic parameters in the population pharmacokinetic model of the compound of formula (I) and propofol.

According to some specific embodiments of the present disclosure, the method for determining the population pharmacokinetic model of the compound of formula (I) and propofol comprises the following steps (the population pharmacokinetic model of the compound of formula (I) and propofol can be determined by using a method comprising the following steps):
(1) acquisition of data;
(2) determination of data included in an analysis;
(3) processing of data;
(4) establishment of a preliminary population pharmacokinetic foundation model;
(5) establishment of a final population pharmacokinetic foundation model;
(6) establishment of the population pharmacokinetic model; and
(7) evaluation of the population pharmacokinetic model.

It can be understood that the serial numbers (1), (2), (3), . . . etc. before the above-mentioned steps of the present disclosure should be understood as the numbers of various steps, and should not be understood as a limitation on the order of the steps.

According to some specific embodiments of the present disclosure, the above-mentioned steps can be performed sequentially according to the order as described above.

According to some specific embodiments of the present disclosure, the data in step (1) are derived from clinical trial data.

According to some specific embodiments of the present disclosure, step (2) comprises determining a pharmacokinetic data set included in the analysis by evaluating the clinical trial data included.

According to some specific embodiments of the present disclosure, the data included in the analysis in step (2) comprise plasma drug concentration data, baseline demographic characteristic data, blood biochemical index data and blood collection sites.

According to some specific embodiments of the present disclosure, the baseline demographic characteristic data comprise any combination of two or more of race, age, height, weight and gender; and the blood biochemical index data comprise any combination of two or more of blood total protein content, creatinine clearance, glutamic-oxalacetic transaminase, glutamic-pyruvic transaminase, alkaline phosphatase and total bilirubin.

According to some specific embodiments of the present disclosure, step (2) comprises determining a pharmacokinetic data set included in the analysis by evaluating the clinical trial data included.

According to some specific embodiments of the present disclosure, step (3) comprises determining and processing one or a combination of two or more of observations below a lower limit of detection, anomalous value data, outliers and missing covariates.

According to some specific embodiments of the present disclosure,
determining and processing the observations below the lower limit of detection comprises: determining the lower limit of detection by a detecting instrument, wherein the observations below the lower limit of detection are not used for population pharmacokinetic analysis, and if the proportion of the observations below the lower limit of detection is greater than 15%, investigating the influence of the observations below the lower limit of detection on model goodness-of-fit and modeling parameters by using a likelihood function method;
determining and processing the anomalous value data comprises: checking whether there are anomalous values in a sample according to a plasma concentration—time curve in subjects receiving drug administration, and eliminating the anomalous values;
determining and processing the outliers comprises: determining outliers according to residual analysis of preliminary modeling results, and eliminating the outliers; and
processing the missing covariates comprises: if the missing covariate for the subject is less than 15%, applying imputation with the median of the data set for continuous covariates, and applying imputation with a value of the most common category for categorical covariates; and if the missing covariate for the subject is greater than 15%, applying no imputation, and performing exploratory analysis on the PK parameters of the subject with full covariate information by using a Bayesian estimation method.

According to some specific embodiments of the present disclosure, the data lower than LLOQ in pharmacokinetic samples only account for 6.8% (167/2463), so a M3 method does not need to be used.

According to some specific embodiments of the present disclosure, the anomalous value data comprise:
1) repeated concentration records at the same time point, except for concentrations in the artery and the vein at the same time point;
2) a trough concentration greater than a corresponding peak concentration;
3) an administration time point after the peak concentration;
4) an administration time point before the trough concentration;
5) concentration records after the end of intravenous administration and before the peak concentration; and
6) unexplained sudden drop or sudden rise in concentrations.

According to some specific embodiments of the present disclosure, for a method for confirming details of outliers, reference is made to the standard PopPK guidelines (Guidance for Industry: Population Pharmacokinetics issued by the U.S. Food and Drug Administration (FDA) and Guideline on Reporting the Results of Population Pharmacokinetic Analysis issued by the Committee for Medicinal Products for Human Use (CHMP)).

According to some specific embodiments of the present disclosure, the outliers are data points outside the specification range of a data set and are determined according to the residual analysis of the preliminary modeling results.

According to some specific embodiments of the present disclosure, if an absolute value of a conditional weighted residual (CWRES) is greater than 5, the data point is regarded as an outlier and deleted from PopPK modeling. After the final population pharmacokinetic model is determined, the outliers are added to the analysis data for reconstruction of a model, so as to evaluate whether the outliers have an influence on the model.

According to some specific embodiments of the present disclosure, step (4) comprises comparing various structural models on the basis of a plasma drug concentration-time curve, wherein the optimal model is selected as a preliminary structural model, to form a preliminary foundation model with a residual model.

According to some specific embodiments of the present disclosure, the preliminary structural model is a three-compartment model (as shown in FIG. 1) with zero-order absorption and first-order linear elimination from the central compartment, and parameters of the preliminary structural model comprise:
central compartment clearance, CL; volume of distribution in the central compartment, V1; volume of distribution in the peripheral compartment 1, V2; volume of distribution in the peripheral compartment 2, V3; intercompartmental clearance between the peripheral compartment 1 and the central compartment, Q2; intercompartmental clearance between the peripheral compartment 2 and the central compartment, Q3; infusion rate, R0; and elimination rate constant K.

According to some specific embodiments of the present disclosure, with the preliminary foundation model, interindividual variation of the PK parameters is described by using the following equation:

$${θ}_{i} = exp ( {θ}_{T} + {η}_{i} )$$

wherein θi represents a PK parameter of the ith subject; θ_{T} represents a natural logarithm of a population typical value of the PK parameter; and ηᵢ represents interindividual variation, and is a random variable following a normal distribution with mean 0 and variance ω², wherein the ω² value represents a diagonal element of a variance-covariance matrix of the interindividual variation.

According to some specific embodiments of the present disclosure, with the preliminary foundation model, variability of a residual is described by using the following equation:

$$log ( {y}_{ij} ) = log ( {\hat{y}}_{ij} ) + {ε}_{ij}$$

wherein yᵢⱼ represents the jth observed concentration of the ith subject, ŷᵢⱼ represents the jth model-predicted concentration of the ith subject, and εᵢⱼ represents a proportional residual of the jth observed concentration of the ith subject, wherein the observed concentration and the predicted concentration are independent of each other and follow a normal distribution with mean 0 and variance σ2, respectively.

According to some specific embodiments of the present disclosure, step (4) comprises comparing any two or more selected from the structural models of a one-compartment model, a two-compartment model and a three-compartment model on the basis of the plasma drug concentration-time curve, and selecting the best preliminary structural model.

According to some specific embodiments of the present disclosure, step (4) comprises comparing various structural models by comparing whether the decrease of objective function values of two nested models is significant, and selecting the best preliminary structural model.

According to some specific embodiments of the present disclosure, step (5) comprises determining covariates included in the evaluation on the basis of clinical knowledge and a drug action mechanism, and establishing a final population pharmacokinetic foundation model on the basis of the covariates included in the evaluation.

According to some specific embodiments of the present disclosure, the covariates included in the evaluation comprise baseline demographic characteristic covariates, blood biochemical index covariates and blood collection sites.

According to some specific embodiments of the present disclosure, the baseline demographic characteristic covariates comprise any combination of two or more of baseline values for age, gender, weight and race; and the blood biochemical index covariates comprise any combination of two or more of creatinine clearance, total protein, glutamic-oxalacetic transaminase, glutamic-pyruvic transaminase, alkaline phosphatase and total bilirubin.

According to some specific embodiments of the present disclosure, step (6) comprises:
a) pre-screening of covariates; and
b) final screening of the covariates by using a forward method and a backward method, and establishing the population pharmacokinetic model.

According to some specific embodiments of the present disclosure, the pre-screening of the covariates comprises:
analyzing the correlation between the PK parameters and each covariate by a graphic method, using linear regression for continuous covariates and using an analysis of variance test for categorical covariates; evaluating the data set on the basis of the model; estimating the parameters of the subject with the final foundation model by using a Bayesian estimation method; and estimating the influence of the covariates on the PK parameters.

According to some specific embodiments of the present disclosure, the pre-screening of the covariates comprises:
analyzing the correlation between the continuous covariates and the PK parameters by using the following equation:

$${θ}_{i} = {θ}_{pop} \cdot {( \frac{{Cov}_{i}}{{Cov}_{pop}} )}^{{k}_{cov}} ;$$

and analyzing the correlation between the categorical covariates and the PK parameters by using the following equation:

$${θ}_{i} = {θ}_{pop} \times exp ( {k}_{c ov} \cdot {X}_{i} )$$

wherein θᵢ represents a PK parameter of the ith subject; θₚₒₚ represents a test population typical value of the PK parameter; Covᵢ represents a continuous covariate of the ith subject; Covₚₒₚ represents a median of the continuous variables in a test population; Xᵢ represents a categorical variable index of the ith subject, wherein value 0 represents the category with the most common category of a covariate, while other integer values represent other categories respectively; and K_{cov} represents a coefficient describing the influence of the covariate.

According to some specific embodiments of the present disclosure, the final screening of the covariates comprises:
establishing a full model by using a forward method on the basis of the final foundation model, and then establishing the population pharmacokinetic model by using a backward method on the basis of the full model, wherein
the forward method comprises: sequentially adding each covariate to a preliminary structural model of the final foundation model in step (5), wherein with a log likelihood ratio test, if an objective function value is decreased by more than 6.63 after adding 1 covariate, the newly added covariate is considered significant, with p<0.01; and firstly adding the covariate having the most significant influence (the more the objective function value decreases, the more significant the influence is, and the most significant influence means that the covariate has the largest decrease in the objective function value) on the basis of the preliminary structural model to form an improved model, then testing the statistically significant covariate screened in the previous step with the improved model, and repeating the process until no significant covariates can be found; and
the backward method comprises: a process of deleting the covariates one by one on the basis of the full model, wherein if an objective function value is increased by more than 10.83 after deleting 1 covariate, the deleted covariate is considered significant, with p<0.001.

The forward method: In order to establish a full model, covariates are sequentially added to the foundation model by using a stepwise forward addition method. The covariate having the most significant influence is firstly added on the basis of the structural model to form an improved model, then the statistically significant covariate screened in the previous step is tested in the improved model, and the process is repeated until no significant covariates can be found. In the forward method, if there are highly correlated covariates, covariates of clinical significance are investigated firstly according to the actual situation, and the covariates highly correlated therewith are investigated in the last step.

The backward method: This method is a process of deleting the covariates one by one on the basis of the full model.

According to some specific embodiments of the present disclosure, step (7) comprises evaluating the population pharmacokinetic model by using one or a combination of two or more of the following methods: a model goodness-of-fit (GOF) diagnostic plot, a visual predictive check (pcVPC), bootstrap and shrinkage.

The bootstrap is a resampling technique, i.c., nonparametric bootstrap, to evaluate the stability of the model.

According to some specific embodiments of the present disclosure,
the model goodness-of-fit diagnostic plot comprises one or a combination of two or more of the following figures: a relation diagram of a population predicted concentration (PRED) and an observed concentration (DV), a relation diagram of an individual predicted concentration (IPRED) and an observed concentration, a relation diagram of a conditional weighted residual (CWRES) and a population predicted concentration, and a relation diagram of a conditional weighted residual and time after first administration, wherein
the visual predictive check (prediction-corrected visual predictive check) comprises simulating 1000 trials according to final model parameters, covariates and actual dosages of administration, and comparative mapping of a predicted result and a measured value so as to evaluate whether the population pharmacokinetic model can well describe a plasma drug concentration-time curve of a propofol derivative;
the bootstrap comprises repeatedly fitting with population pharmacokinetic model until 1000 data sets for bootstrap replication, and randomly selecting subject data and covariates (including concentrations, time points, administration history and all covariates) for replacement to achieve the replication (the number of subjects in each data set is the same as that in the original data set); and
the shrinkage comprises estimating individual parameter values of a subject by using a Bayesian estimation method with the population pharmacokinetic model, and calculating interindividual variations and individual residuals from model predictions and observations.

The visual predictive check can be used to compare the consistency of the observations and the model-predicted median of the plasma drug concentration-time curve and the distribution range (2.5th to 97.5th percentile).

The shrinkage is used to evaluate the interindividual variations (Ω) and residuals (ε) of the final model, and quantify the individual parameter values and random error estimates. If the shrinkage values of @ and & are large, such as >30%, the Bayesian estimation method should be considered carefully.

According to some specific embodiments of the present disclosure, the shrinkage comprises evaluating the interindividual variations and residuals of the pharmacokinetic parameters by using the following equation, and quantifying the individual parameter values and random error estimates:

$${η}_{shrinkage} = 1 - \frac{SD ( {\hat{η}}_{p h} )}{ω}$$

$${ε}_{shrinkage} = 1 - SD ( IWRES )$$

wherein η_{shrinkage} is interindividual variation, ε_{shrinkage} is an individual residual, ω is interindividual variation degree of individual parameter values estimated by the population pharmacokinetic model, ηₚₕ is η value of the parameter for all individuals, IWRES is an individual weighted residual; and SD represents standard deviation.

According to some specific embodiments of the present disclosure, step (7) further comprises estimating individual PK parameters of the subject by using a Bayesian post-hoc method, simulating a plasma drug concentration-time curve of intravenous infusion according to actual dosages of administration, and calculating the area under the plasma drug concentration-time curve from 0-1 min (AUC 0-1 min), the area under the plasma drug concentration-time curve from 0-2 min (AUC 0-2 min), the area under the plasma drug concentration-time curve from 0-4 min (AUC 0-4 min), the area under the plasma drug concentration-time curve from 0-10 min (AUC 0-10 min), the area under the plasma drug concentration-time curve from 0-24 h (AUC 0-24 h) and the peak concentration (Cmax).

According to some specific embodiments of the present disclosure, clinical trials included in the analysis of the present disclosure comprise:
propofol derivative SAD: a phase I ramp-up trial of single intravenous injection of placebo and positive drug control in Australian healthy subjects;
propofol derivative SAD_02: a phase I ramp-up trial of single intravenous injection of positive drug control in Australian healthy subjects;
propofol derivative SAD_03: a safety and tolerance trial of single intravenous injection plus 30 min continuous intravenous infusion in Australian subjects;
propofol derivate-101: a single-center, open-label, uncontrolled phase I ramp-up trial for evaluating the single intravenous injection of propofol derivative injectable emulsion in Chinese healthy subjects;
propofol derivate-103: a single-center, open-label, randomized, two-phase, crossover study for evaluating the interaction of propofol derivative injectable emulsion and rifampin capsules (DDI) in Chinese healthy subjects;
propofol derivate-202: a multi-center, open-label, non-randomized, positive-control, dose-ramping phase IIa clinical study for evaluating the tolerance, efficacy and safety of propofol derivative injectable emulsion for induction of general anesthesia in patients undergoing elective surgery; and
propofol derivate-302: a multi-center, randomized, double-blind, propofol-controlled, parallel-group phase III study for evaluating the efficacy and safety of the propofol derivative in induction of general anesthesia in Chinese subjects undergoing elective surgery as compared with propofol.

According to some specific embodiments of the present disclosure, when at least one well-recorded administration time and a corresponding plasma concentration are collected from a subject after administration, the subject can be included in the population pharmacokinetic analysis.

According to some specific embodiments of the present disclosure, the method of the present disclosure is a method for determining a population pharmacokinetic model of propofol derivative injectable emulsion.

According to some specific embodiments of the present disclosure, the components of the propofol derivative injectable emulsion comprise: soybean oil, glycerol, triglyceride, egg yolk lecithin, sodium oleate and sodium hydroxide (see WO/2016/034079).

According to some specific embodiments of the present disclosure, the PopPK analysis method is based on the Guidance for Industry: Population Pharmacokinetics issued by the U.S. Food and Drug Administration (FDA) and the Guideline on Reporting the Results of Population Pharmacokinetic Analysis issued by the Committee for Medicinal Products for Human Use (CHMP).

According to some specific embodiments of the present disclosure, the PopPK analysis method uses a nonlinear mixed effect model (NONMEM) method to estimate typical values of the parameters and variations thereof.

According to some specific embodiments of the present disclosure, the PopPK analysis software used in the present disclosure is NONMEM 7, version 7.4.0 (ICON Development Solutions. Ellicott City, Maryland, USA); and Perl Speaks NONMEM (PsN), version 3.2.12 (Uppsala University, Sweden).

The structural models of the present disclosure are ultimately selected from a three-compartment model with zero-order absorption and first-order linear elimination from the central compartment, as shown in FIG. 1. The PopPK model is composed of the following parameters: central compartment clearance (CL), volume of distribution in the central compartment (V1), volumes of distribution in the peripheral compartments (V2, V3), and intercompartmental clearances (Q2, Q3).

$$\frac{{dX}_{1}}{dt} = {R}_{0} - ( k + {k}_{12} + {k}_{13} ) \cdot {X}_{1} + {k}_{21} \cdot {X}_{2} + {k}_{31} \cdot {X}_{3}$$

$$\frac{{dX}_{2}}{dt} = {k}_{12} \cdot {X}_{1} - {k}_{21} \cdot {X}_{2}$$

$$\frac{{dX}_{3}}{dt} = {k}_{13} \cdot {X}_{1} - {k}_{31} \cdot {X}_{3}$$

$${R}_{0} = Infusion rate$$

$$k = Emination rate constant k = CL / {V}_{1}$$

$${k}_{12} = Intercompartmental rate constant between the central compartment and the peripheral compartment 1 {k}_{12} = {Q}_{2} / {V}_{1}$$

$${k}_{21} = Intercompartmental rate constant between the peripheral compartment 1 and the central compartment {k}_{21} = {Q}_{2} / {V}_{2}$$

$${k}_{22} = Intercompartmental rate constant between the central compartment and the peripheral compartment 1 {k}_{13} = {Q}_{3} / {V}_{1}$$

$${k}_{31} = Intercompartmental rate constant between the peripheral compartment 1 and the central compartment {k}_{31} = {Q}_{1} / {V}_{1}$$

$${X}_{1} = Drug amount in the central compartment$$

$${X}_{2} = Drug amount in the peripheral compartment 1$$

$${X}_{3} = Drug amount in the peripheral compartment 2$$

$$CL = Central compartment clearan$$

$${V}_{1} = Volume of distribution in the central compartment$$

$${Q}_{2} , {Q}_{3} = Intercompartmental clearance$$

$${V}_{2} , {V}_{3} = Volume of distribution in the peripheral compartment$$

The parameters are estimated by using the FOCEI method of NONMEM. The correlation between random effects in the PK base model is estimated by using a diagonal Ω matrix. On the basis of the model diagnostics, a proportional model is selected as a residual model.

According to some specific embodiments of the present disclosure, the pre-screening results of step (5) show that:
the following covariates all have a significant influence (p<0.01) on the PK parameters, are included in a covariate model screening process, and investigated by using a forward method and a backward method:
(1) central compartment clearance CL: creatinine clearance (CLCR), total protein (TP), total bilirubin (TBIL), weight (WT), age (AGE), race (RACE) and blood collection site (SITE);
(2) volume of distribution in the central compartment V1: age (AGE), race (RACE) and blood collection site (SITE).

According to some specific embodiments of the present disclosure, a stepwise forward addition method (the forward method) is used for the PopPK model in NONMEM. The results show that WT, TP and SITE have a significant influence (p<0.01) on CL, AGE has a significant influence (p<0.01) on V1, and it is not found that RACE has a significant influence on CL or V1 by investigating RACE on this basis. By using a stepwise backward elimination method (the backward method), no influence factors are eliminated (Δ−2LL>10.83).

In another aspect, the present disclosure further provides a system for determining individual administration parameters of a compound of formula (I) or propofol.

The administration parameters can comprise individual administration data, specifically, such as dosage of administration. More specifically, the dosage of administration can comprise single dosage of administration, daily dosage of administration, administration time, administration frequency, etc. of an individual.

According to some specific embodiments of the present disclosure, the system for determining the individual administration parameters of the compound of formula (I) comprises a data acquisition device, a data processing device and a result output device,

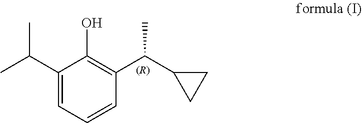

wherein the data comprise baseline demographic characteristic data, blood biochemical index data and blood collection site information data; with the data processing device, individual administration parameter results of the compound of formula (I) are obtained by using the following equation:

$${CL}_{i} = exp ( 4.2 + 0.349 \cdot log ( WT / 63.9 ) - 0.749 \cdot log ( TP / 72.4 ) + 0.238 \cdot SITE + {η}_{CL , i} ) ;$$

wherein CLᵢ represents central compartment clearance of the ith subject; when collecting a sample from venous blood, SITE=0, and when collecting a sample from artery blood, SITE=1; WT represents weight; TP represents total protein; η_{CL,i} is interindividual variation of the CL of the ith subject; and η follows a normal distribution with mean 0 and variance ω2, wherein the ω2 is a diagonal element of a variance-covariance matrix 22 of the interindividual variation.

According to some specific embodiments of the present disclosure, the result output device is used to output individual administration parameter results.

According to some specific embodiments of the present disclosure, with the data processing device, individual administration parameter results of the compound of formula (I) are obtained by using the following equation:

$${CL}_{i} = exp ( 4.2 + 0.349 \cdot log ( WT / 63.9 ) - 0.749 \cdot log ( TP / 72.4 ) + 0.238 \cdot SITE + {η}_{CL , i} )$$

$${V}_{1 i} = exp ( 0.908 + 0.426 \cdot log ( AGE / 27 ) + {η}_{{V}_{1} , i} )$$

$${Q}_{2 i} = exp ( 4.06 + {η}_{{Q}_{2} , i} )$$

$${V}_{2 i} = exp ( 1.75 )$$

$${Q}_{3 i} = exp ( 4.08 + {η}_{{Q}_{3} , i} )$$

$${V}_{3 i} = exp ( 4.36 + {η}_{{V}_{3} , i} ) ;$$

wherein V₁ᵢ represents volume of distribution in the central compartment of the ith subject;
V₂ᵢ represents volume of distribution in the peripheral compartment 1 of the ith subject;
V₃ᵢ represents volume of distribution in the peripheral compartment 2 of the ith subject;
Q₂ᵢ represents intercompartmental clearance between the peripheral compartment 1 and the central compartment of the ith subject;
Q₃ᵢ represents intercompartmental clearance between the peripheral compartment 2 and the central compartment of the ith subject;
AGE represents age; and η represents interindividual variation of a corresponding parameter.

According to some specific embodiments of the present disclosure, the system for determining the individual administration parameters of propofol comprises a data acquisition device, a data processing device and a result output device, wherein the data comprise baseline demographic characteristic data, blood biochemical index data and blood collection site information data; with the data processing device, individual administration parameter results of propofol are obtained by using the following equation:

$${CL}_{i} = exp ( 4.56 + {η}_{CL , i} )$$

wherein CLᵢ represents central compartment clearance of the ith subject; η_{CL,i} is interindividual variation of the CL of the ith subject; and η follows a normal distribution with mean 0 and variance ω2, wherein the ω2 is a diagonal element of a variance-covariance matrix Ω of the interindividual variation.

According to some specific embodiments of the present disclosure, with the data processing device, individual administration parameter results of propofol are obtained by using the following equation:

$${CL}_{i} = exp ( 4.56 + {η}_{CL , i} )$$

$${V}_{1 i} = exp ( 2.25 + {η}_{{V}_{1} , i} )$$

$${Q}_{2 i} = exp ( 4.96 )$$

$${V}_{2 i} = exp ( 3.63 )$$

$${Q}_{3 i} = exp ( 3.88 )$$

$${V}_{3 i} = exp ( 5.57 )$$

wherein V₁ᵢ represents volume of distribution in the central compartment of the ith subject;
V₂ᵢ represents volume of distribution in the peripheral compartment 1 of the ith subject;
V₃ᵢ represents volume of distribution in the peripheral compartment 2 of the ith subject;
Q₂ᵢ represents intercompartmental clearance between the peripheral compartment 1 and the central compartment of the ith subject;
Q₃ᵢ represents intercompartmental clearance between the peripheral compartment 2 and the central compartment of the ith subject;
AGE represents age; and η represents interindividual variation of a corresponding parameter.

According to some specific embodiments of the present disclosure, the result output device is used to output individual administration parameter results.

The methods not specified in the present application can be implemented with reference to conventional methods in the field.

In conclusion, the present disclosure provides a method and system for determining a population pharmacokinetic model of a propofol derivative and propofol. The method of the present disclosure has the following advantages:

The three-compartment linear pharmacokinetic model can well describe the pharmacokinetic characteristics in the dosage range in this study. After model evaluation, it shows that the final model has relatively good stability, accuracy and good prediction performance, no obvious deviation is observed in the diagnostic plot, and the pcVPC result shows that the model can fully reproduce the central tendency and variability of the original data. The population pharmacokinetic model of the propofol derivative and propofol established by the method of the present disclosure can be used for guiding the dosage regimen in the following clinical trials (phase I, phase II or phase III).

The weight and the total protein show a significant influence on the CL of the drug. The weight in the range of 45 kg to 90 kg shows a relatively small influence (the relative median changes are −11.6% and 12.7%, respectively). The influence of the total protein (the relative median changes are 6.8% and −6.3%, respectively) is expected to be clinically insignificant. CL estimated by the model is also significantly influenced by different blood collection sites. The age has a significant influence on the V1 of the drug, and the results in the trials show that the age in the range of 19.8 to 53 years old has no influence on AUC0-24 and has an influence on Cmax, but is expected to be clinically insignificant.

### BRIEF DESCRIPTION OF THE DRAWINGS

FIG. 1 shows a structural diagram of a three-compartment model of the present disclosure;

FIG. 2 shows diagnostic plots of a final drug model in example 1 of the present disclosure;

FIG. 3 shows distribution diagrams of interindividual variations (ETA) of the final model of the drug in example 1 of the present disclosure;

FIG. 4 shows prediction-corrected visual predictive check (pcVPC) plots of the drug in example 1 of the present disclosure;

FIG. 5 shows diagnostic plots of a final drug model in example 2 of the present disclosure;

FIG. 6 shows prediction-corrected visual predictive check (pcVPC) plots of the drug in example 2 of the present disclosure;

FIG. 7 shows a plasma drug concentration-time relational plot of the drug in example 1 of the present disclosure;

FIG. 8 shows a plasma drug concentration-time relational plot of the drug in example 2 of the present disclosure.

### DETAILED DESCRIPTION OF EMBODIMENTS

The technical solutions of the present disclosure will be described in detail below in conjunction with the drawings and examples, but the protection scope of the present disclosure includes but is not limited thereto.

### EXAMPLE 1

This example provides a method for determining a population pharmacokinetic model of a propofol derivative. The method comprises:

### 1. Acquisition of Data: Clinical Trial

propofol derivative SAD: a phase I ramp-up trial of single intravenous injection of placebo and positive drug control in Australian healthy subjects;
propofol derivative SAD_02: a phase I ramp-up trial of single intravenous injection of positive drug control in Australian healthy subjects;
propofol derivative SAD_03: a safety and tolerance trial of single intravenous injection plus 30 min continuous intravenous infusion in Australian subjects;
propofol derivate-101: a single-center, open-label, uncontrolled phase I ramp-up trial for evaluating the single intravenous injection of propofol derivative injectable emulsion in Chinese healthy subjects;
propofol derivate-103: a single-center, open-label, randomized, two-phase, crossover study for evaluating the interaction of propofol derivative injectable emulsion and rifampin capsules (DDI) in Chinese healthy subjects;
propofol derivate-202: a multi-center, open-label, non-randomized, positive-control, dose-ramping phase IIa clinical study for evaluating the tolerance, efficacy and safety of propofol derivative injectable emulsion for induction of general anesthesia in patients undergoing elective surgery; and
propofol derivate-302: a multi-center, randomized, double-blind, propofol-controlled, parallel-group phase III study for evaluating the efficacy and safety of the propofol derivative in induction of general anesthesia in Chinese subjects undergoing elective surgery as compared with propofol.

### 2. Determination of Data Included in an Analysis

The sampling scheme is as shown in Table 1 below:

TABLE 1
Sampling scheme

| Scheme no. | Dosage | Planned sampling design |
|---|---|---|
| Propofol | 0.128 mg/kg | Arterial blood samples were collected within 30 min (0 min) |
| derivative SAD | 0.192 mg/kg | before injection and at 0.5 min, 1 min, 1.5 min, 2 min, 2.5 |
| | 0.288 mg/kg | min, 3 min, 3.5 min, 4 min, 5 min, 6 min, 8 min, 12 min, 15 |
| | 0.432 mg/kg | min, 30 min, 60 min, 90 min, 2 hr, 3 hr and 4 hr after the start |
| | 0.54 mg/kg | of injection; and venous blood samples were collected at 6 hr, |
| | 0.648 mg/kg | 8 hr and 24 hr after the start of injection. |
| | 0.81 mg/kg | |
| Propofol | 0.288 mg/kg | Arterial blood samples were collected within 30 min (0 min) |
| derivative | 0.432 mg/kg | before injection and at 0.5 min, 1 min, 1.5 min, 2 min, 2.5 |
| SAD_02 | 0.54 mg/kg | min, 3 min, 3.5 min, 4 min, 5 min, 6 min, 8 min, 12 min, 15 |
| | 0.648 mg/kg | min, 30 min, 60 min, 90 min, 2 hr, 3 hr and 4 hr after the start |
| | 0.81 mg/kg | of injection; and venous blood samples were collected at 6 hr, |
| | | 8 hr and 24 hr after the start of injection. |
| Propofol | 0.288 mg/kg + 1 | Arterial blood samples were collected within 30 min (0 min) |
| derivative | mg/kg/h | before injection and at 0.5 min, 1 min, 1.5 min, 2 min, 2.5 |
| SAD_03 | 0.540 mg/kg + 2 | min, 3 min, 3.5 min, 4 min, 5 min, 8 min, 11 min, 21 min, 25 |
| | mg/kg/h | min, 31 min, 32 min, 34 min, 36 min, 40 min, 50 min, 60 min, |
| | | 90 min, 2 hr, 3 hr and 4 hr after the start of first injection; and |
| | | venous blood samples were collected at 6 hr, 8 hr and 24 hr |
| | | after the start of first injection. |
| Propofol | 0.15 mg/kg | Venous blood samples were collected within 30 min (0 min) |
| derivate-101 | 0.4 mg/kg | before injection, at 0.5 min, 1 min, 2 min, 3 min, 5 min, 8 min, |
| | 0.6 mg/kg | 15 min, 30 min, 60 min, 90 min, 2 hr, 3 hr, 4 hr, 6 hr, 8 hr and |
| | 0.9 mg/kg | 24 hr after the start of injection, and at time points when the |
| | | verbal response disappeared and recovered. |
| Propofol | 0.4 mg/kg | Arterial blood samples were collected within 30 min (0 min) |
| derivate-103 | 0.4 mg/kg + rifampin | before injection and at 1 min, 2 min, 4 min, 8 min, 15 min, 30 |
| | | min and 60 min after the start of injection; and venous blood |
| | | samples were collected at 2 hr, 3 hr, 4 hr, 6 hr, 8 hr, 12 hr and |
| | | 24 hr after the start of injection. |
| Propofol | 0.3 mg/kg | Only venous blood samples were collected within 30 min (0 |
| derivate-202 | 0.4 mg/kg | min) before injection and at 0.5 min, 1 min, 2 min, and 3 min |
| | 0.5 mg/kg | after the start of injection, except for 4 cases in which both |
| | | arterial and venous blood samples were collected. |
| Propofol | Propofol derivatives: | Venous blood samples were collected within 30 min (0 min) |
| derivate-302 | 0.4 mg/kg (first time) | before injection and at 5 min, 15 min and 60 min after the start |
| | 0.2 mg/kg | of injection. |
| | (supplementing if | |
| | necessary) | |

The original data set comprises 2609 measurable plasma drug concentration data from 219 subjects (wherein 81 people receive both arterial and venous blood collection, and subjects receiving another blood collecting method are regarded as new individuals, that is, 300 individuals are included in the population analysis).

A pharmacokinetic data set included in the analysis are determined by evaluating the clinical trial data included:
plasma drug concentration (see FIG. 7);
baseline demographic characteristic data: race, age, height, weight and gender;
blood biochemical index data: blood total protein content, creatinine clearance, glutamic-oxalacetic transaminase and glutamic-pyruvic transaminase; and
blood collection sites.

### 3. Processing of Data

Processing the data in step (2) comprising:
determining and processing the observations below the lower limit of detection comprising: determining the lower limit of detection by a detecting instrument, wherein the observations below the lower limit of detection are not used for population pharmacokinetic analysis, and if the proportion of the observations below the lower limit of detection is greater than 15%, investigating the influence of the observations below the lower limit of detection on model goodness-of-fit and modeling parameters by using a likelihood function method;
determining and processing the anomalous value data comprising: checking whether there are anomalous values in a sample according to a plasma concentration—time curve in subjects receiving drug administration, and eliminating the anomalous values;
determining and processing the outliers comprising: determining outliers according to residual analysis of preliminary modeling results, and eliminating the outliers; and
processing the missing covariates comprising: if the missing covariate for the subject is less than 15%, applying imputation with the median of the data set for continuous covariates, and applying imputation with a value of the most common category for categorical covariates; and if the missing covariate for the subject is greater than 15%, applying no imputation, and performing exploratory analysis on the PK parameters of the subject with full covariate information by using a Bayesian estimation method.

### 4. Establishment of a Preliminary Population Pharmacokinetic Foundation Model

Establishing the preliminary foundation model on the basis of the data processed in step 3 comprising:
(1) describing interindividual variation of the PK parameters by using the following equation:

$${θ}_{i} = exp ( {θ}_{T} + {η}_{i} ) ;$$

(2) describing variability of a residual by using the following equation:

$$log ( {y}_{ij} ) = log ( {\hat{y}}_{ij} ) + {ε}_{ij} ;$$

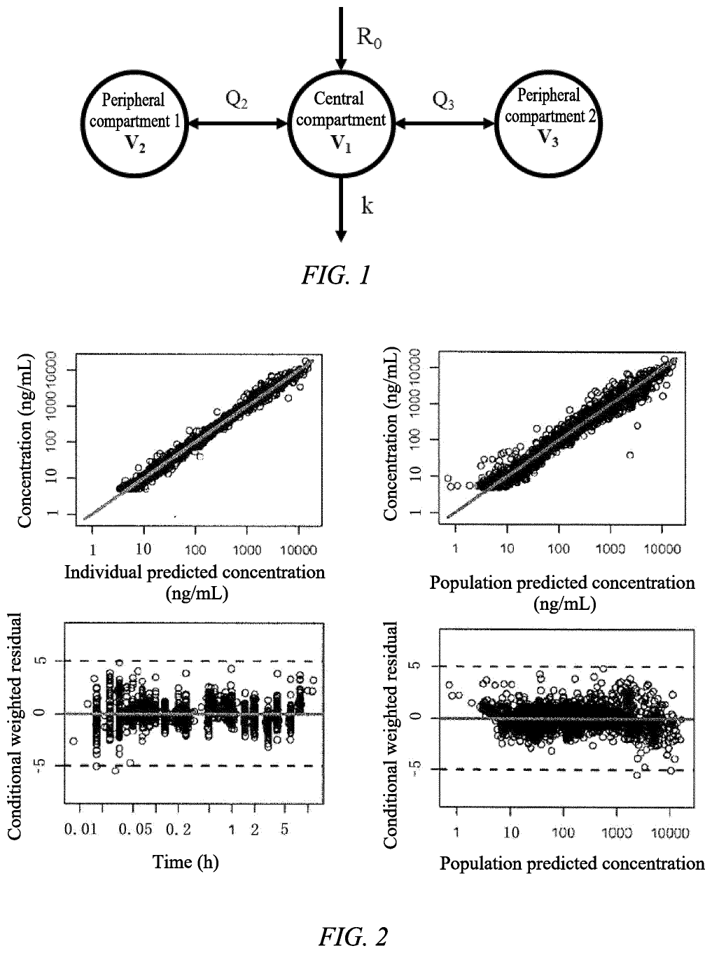

(3) comparing a one-compartment model, a two-compartment model and a three-compartment model by comparing whether the decrease of the objective function values of the two nested models is significant on the basis of the plasma drug concentration-time curve, and selecting the three-compartment model as shown in FIG. 1 as the preliminary foundation model, wherein CL represents central compartment clearance; V1 represents volume of distribution in the central compartment; V2 represents volume of distribution in the peripheral compartment 1; V3 represents volume of distribution in the peripheral compartment 2; Q2 represents intercompartmental clearance between the peripheral compartment 1 and the central compartment; Q3 represents intercompartmental clearance between the peripheral compartment 2 and the central compartment; R0 represents infusion rate; and K represents elimination rate constant.

### 5. Establishment of a Final Population Pharmacokinetic Foundation Model

Establishing the final foundation model on the basis of the preliminary foundation model in step 4 comprising:
(1) determining covariates included in the evaluation on the basis of clinical knowledge and a drug action mechanism:
baseline demographic characteristic covariates: baseline values for age, gender, weight and race;
blood biochemical index covariates: creatinine clearance, total protein, glutamic-oxalacetic transaminase, glutamic-pyruvic transaminase, alkaline phosphatase and total bilirubin; and
blood collection sites.
(2) establishing a final population pharmacokinetic foundation model on the basis of the covariates included in the evaluation.

### 6. Establishment of the Population Pharmacokinetic Model

Establishing the population pharmacokinetic model by adding the screened covariates on the basis of the final foundation model in step 5 comprising:
(1) Pre-screening of covariates:
analyzing the correlation between the continuous covariates and the PK parameters by using the following equation:

$${θ}_{i} = {θ}_{pop} \cdot {( \frac{{Cov}_{i}}{{Cov}_{pop}} )}^{{k}_{co v}} ;$$

and analyzing the correlation between the categorical covariates and the PK parameters by using the following equation:

$${θ}_{i} = {θ}_{pop} \times exp ( {k}_{c ov} \cdot {X}_{i} )$$

(2) Final screening of the covariates by using a forward method and a backward method, and establishing the population pharmacokinetic model:

The forward method: sequentially adding each covariate to a preliminary structural model of the final foundation model in step (5), wherein with a log likelihood ratio test, if an objective function value is decreased by more than 6.63 after adding 1 covariate, the newly added covariate is considered significant, with p<0.01; and firstly adding the covariate having the most significant influence on the basis of the preliminary structural model to form an improved model, then testing the statistically significant covariate screened in the previous step with the improved model, and repeating the process until no significant covariates can be found to obtain a full model;

The backward method: a process of deleting the covariates one by one on the basis of the full model, wherein if an objective function value is increased by more than 10.83 after deleting 1 covariate, the deleted covariate is considered significant, with p<0.001.

A stepwise forward addition method is used for the propofol derivative PopPK model in NONMEM. The results show that WT, TP and SITE have a significant influence (p<0.01) on CL, AGE has a significant influence (p<0.01) on V₁, and it is not found that RACE has a significant influence on CL or V₁ by investigating RACE on this basis. By using a stepwise backward elimination method, no influence factors are eliminated (Δ_{-2LL}>10.83). A stepwise forward addition method is used for the propofol PopPK model in NONMEM. The results show that WT has a significant influence (p<0.01) on CL. The influence of WT on the CL of the propofol derivative is eliminated by using a stepwise backward elimination method (Δ−2LL=8.691).
(3) Establishing the model

$${CL}_{i} = exp ( 4.2 + 0.349 \cdot log ( WT / 63.9 ) - 0.749 \cdot log ( TP / 72.4 ) + 0.238 \cdot SITE + {η}_{CL , i} )$$

$${V}_{1 i} = exp ( 0.908 + 0.426 \cdot log ( AGE / 27 ) + {η}_{{V}_{1} , i} )$$

$${Q}_{2 i} = exp ( 4.06 + {η}_{{Q}_{2} , i} )$$

$${V}_{2 i} = exp ( 1.75 )$$

$${Q}_{3 i} = exp ( 4.08 + {η}_{{Q}_{3} , i} )$$

$${V}_{3 i} = exp ( 4.36 + {η}_{{V}_{3} , i} )$$

In the final PopPK model of the propofol derivative, the weight and the total protein have a significant influence on the CL, and CL estimated by the model is also significantly influenced by different blood collection sites; and the age has an influence on V₁.

### 7. Evaluation of the Population Pharmacokinetic Model

Evaluating the pharmacokinetic model established in step 6 comprising:
(1) Model goodness-of-fit (GOF) diagnostic plot

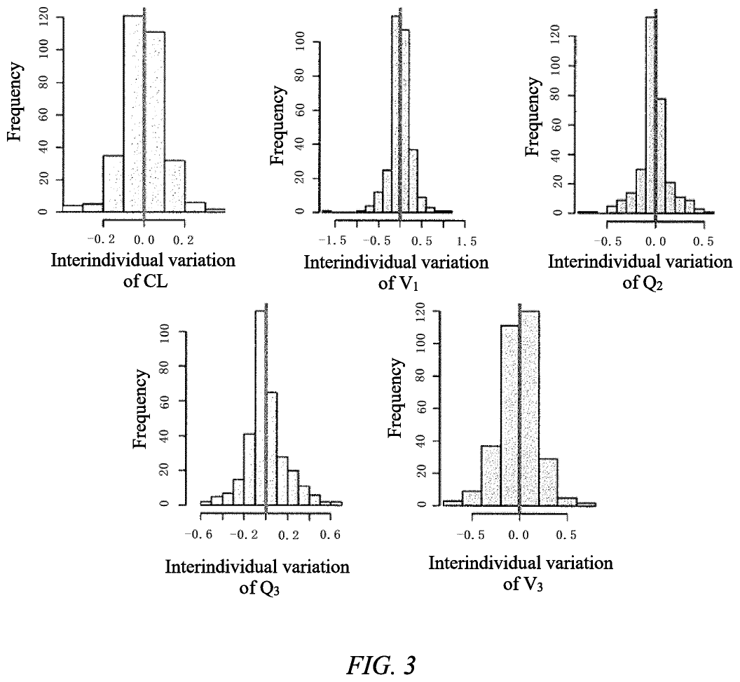

The diagnostic plots of the final PopPK model for the drug are as shown in FIG. 2. The results show that there is good consistency between an observed concentration value and a predicted concentration, and no significant deviations are observed in the plots of conditional weighted residuals versus time and versus a predicted concentration. In FIG. 2, the upper left panel is a diagnostic plot of observations versus individual predictions in the final model of the drug, the upper right panel is a diagnostic plot of observations versus population predictions in the final model of the drug, the lower left panel is a diagnostic plot of conditional weight residuals (CWRES) versus time, and the lower right panel is a diagnostic plot of CWRES versus population predictions, wherein solid lines in the middle are standard lines of 0, and dotted lines are auxiliary lines of |CWRES|=5.
(2) Distribution of interindividual random effects (ETA) of the PopPK final model of the drug is as shown in FIG. 3. The results show that the ETA is generally distributed symmetrically around 0. In FIG. 3, the vertical coordinate is frequency, etaCL is interindividual variation of CL, etaV1 is interindividual variation of V1, etaQ2 is interindividual variation of Q2, etaQ3 is interindividual variation of Q3, etaV3 is interindividual variation of V3, and bold lines in the middle are standard lines of 0.
(3) Prediction-corrected visual predictive check (pcVPC)

pcVPC is used to evaluate the ability of the model to reproduce the data distribution. 1000 trials are simulated and reproduced according to the observed covariate information of each subject, the estimated values of the final population pharmacokinetic model parameters, the random effects and the residual errors. The pc VPC of the plasma drug concentration-time curve is as shown in FIG. 4. FIG. 4 shows the observed concentrations (dots) of all subjects, the model-predicted 95% confidence (middle shaded arca) interval for the median and the model-predicted 95% confidence intervals (upper and lower shaded areas) for the 2.5th and 97.5th percentiles. The results show that the final population pharmacokinetic model can fully predict the central tendency and variability of drug concentrations of all subjects in the clinical study. In FIG. 4, the circles are observations, the middle solid lines are medians of the observations, the upper and lower dashed lines correspond to the 97.5th and 2.5th percentiles respectively, the middle shaded area is the model-predicted 95% confidence interval for the median, the upper and lower shaded areas are 95% confidence intervals for the median lines and the 2.5th and 97.5th percentile lines for data of the 1000 simulations, and data lower than the lower limit of detection are not shown in this figure.
(4) Bootstrap

See Table 2 below for the comparison between the parameters estimated by the final model and the parameters estimated by Bootstrap. The median of the parameters estimated by Bootstrap is similar to the median of the parameters estimated by the final PopPK model. 95% CI of the parameters estimated by the final PopPK model is highly overlapped with 95% CI of the parameters estimated by Bootstrap (represented by the 2.5th to 97.5th percentile interval), indicating that the final model has relatively good stability and accuracy.

TABLE 2
Parameters estimated by final model and parameters estimated by Bootstrap

| | | | Median of Bootstrap |
|---|---|---|---|
| | | | population model |
| | | Typical values of | parameters |
| | | population model | (2.5^{th} to 97.5^{th} |
| Parameters | Parameter description | parameters (95% CI) | percentiles) |
| exp(θ₁) | Clearance, CL (L/hr) | 66.4 (63.5 to 69.2) | 66.4 (63.7 to 69.4) |
| exp(θ₂) | Volume of distribution in the central | 2.48 (2.25 to 2.71) | 2.48 (2.27 to 2.74) |
| | compartment, V₁ (L) | | |
| exp(θ₃) | Intercompartmental clearance | 58 (50.8 to 65.1) | 57.9 (51.5 to 65.6) |
| | between the central compartment and | | |
| | the peripheral compartment 1, Q₂ | | |
| | (L/hr) | | |
| exp(θ₄) | Volume of distribution in the | 5.77 (4.96 to 6.58) | 5.74 (5.07 to 6.7) |
| | peripheral compartment 1, V₂ (L) | | |
| exp(θ₅) | Clearance between the central | 59.0 (54.5 to 63.5) | 59.1 (54.6 to 63.6) |
| | compartment and the peripheral | | |
| | compartment 2, Q₃ (L/hr) | | |
| exp(θ₆) | Volume of distribution in the | 78.3 (72.8 to 83.8) | 78.4 (73 to 83.9) |
| | peripheral compartment 2, V₃ (L) | | |
| θ₇ | Influence of weight on clearance, WT | 0.349 (0.21 to 0.488) | 0.348 (0.206 to 0.489) |
| | on CL | | |
| θ₈ | Influence of age on volume of | 0.426 (0.194 to 0.659) | 0.441 (0.172 to 0.66) |
| | distribution in the central | | |
| | compartment, AGE on V₁ | | |
| θ₉ | Influence of total protein on | −0.749 (−1.21 to −0.285) | −0.743 (−1.22 to −0.259) |
| | clearance, TP on CL | | |
| exp(θ₁₀) | Influence of arterial blood collection | 1.27 (1.2 to 1.34) | 1.27 (1.2 to 1.33) |
| | on clearance, SITE on CL | | |
| ω_{CL} | Interindividual variation of clearance, | 14.5 (12 to 16.9) | 14.3 (11.9 to 17) |
| | CL % | | |
| ω_{V1} | Interindividual variation of volume of | 41.4 (28 to 54.9) | 40.4 (27.7 to 54.5) |
| | distribution in the central | | |
| | compartment, V₁% | | |
| ω_{Q2} | Interindividual variation of | 28.9 (23.1 to 34.7) | 29.0 (23.1 to 35.5) |
| | intercompartmental clearance | | |
| | between the central compartment and | | |
| | the peripheral compartment 1, Q₂% | | |
| ω_{Q3} | Interindividual variation of | 29.1 (23.1 to 35.1) | 28.8 (22.8 to 36.3) |
| | intercompartmental clearance | | |
| | between the central compartment and | | |
| | the peripheral compartment 2, Q₃% | | |
| ω_{V3} | Interindividual variation of volume of | 30.7 (25.6 to 35.8) | 30.5 (25.3 to 35.7) |
| | distribution in the peripheral | | |
| | compartment 2, V₃% | | |
| σ₁ | Residual of venous blood collection | 30.6 (26.6 to 34.5) | 30.5 (26.3 to 34.6) |
| | (%) | | |
| σ₂ | Residual of arterial blood collection | 16.9 (15.8 to 18.1) | 16.9 (15.7 to 18.2) |
| | (%) | | |

(4) Shrinkage

The shrinkage values of the final model parameters are as shown in Table 3.

In the final population pharmacokinetic model. the shrinkage amplitudes of ω_{V1}, ω_{Q2}, ω_{Q3}, and ω_{V3} are slightly higher than 30%.

TABLE 3
Shrinkage values of final model parameters

| | | Shrinkage |
|---|---|---|
| Parameters | Parameter description | values (%) |
| ωCL | Clearance, CL % | 29.2% |
| ωV1 | Volume of distribution in the central | 36.5% |
| | compartment, V1% | |
| ωQ2 | Intercompartmental clearance between the | 44.6% |
| | central compartment and the peripheral | |
| | compartment 1, Q2% | |
| ωQ3 | Intercompartmental clearance between the | 35.5% |
| | central compartment and the peripheral | |
| | compartment 2, Q3% | |
| ωV3 | Volume of distribution in the peripheral | 32.9% |
| | compartment 2, V3% | |
| σ1 | Residual of venous blood collection (%) | 17.3% |
| σ2 | Residual of arterial blood collection (%) | 13.2% |

### Conclusion

The three-compartment linear pharmacokinetic model can well describe the pharmacokinetic characteristics of the propofol derivative in the dosage range in this study. After model evaluation, it shows that the final model has relatively good stability, accuracy and good prediction performance.

The weight and the total protein show a significant influence on the CL of the propofol derivative. The weight in the range of 45 kg to 90 kg shows a relatively small influence (the relative median changes are −11.6% and 12.7%, respectively). The influence of the total protein (the relative median changes are 6.8% and −6.3%, respectively) is expected to be clinically insignificant. CL estimated by the model is also significantly influenced by different blood collection sites. The age has a significant influence on the V₁ of the propofol derivative, and the results in the propofol derivative-302 trial show that the age in the range of 19.8 to 53 years old has no influence on AUC₀₋₂₄ and has an influence on Cmax, but is expected to be clinically insignificant.

### EXAMPLE 2

This example provides a method for determining a population pharmacokinetic model of propofol. The method comprises:

### 1. Acquisition of Data: Clinical Trial

a multi-center, randomized, double-blind, propofol-controlled, parallel-group phase III study for evaluating the efficacy and safety of propofol in induction of general anesthesia in Chinese subjects undergoing elective surgery.

### 2. Determination of Data Included in an Analysis

The sampling scheme is as shown in Table 4 below:

TABLE 4
Propofol sampling scheme

| Scheme | | |
|---|---|---|
| no. | Dosage | Planned sampling design |
| Propofol | Propofol: | Venous blood samples were collected within |
| | 2.0 mg/kg | 30 min (0 min) before injection and at 5 |
| | (first time) | min, 15 min and 60 min after the start |
| | 1.0 mg/kg | of injection. |
| | (supplementing | |
| | if necessary) | |

82 measurable plasma drug concentration data from 28 subjects (60 of 88 subjects treated with propofol in the trial further receive propofol treatment for other purposes after induced anesthesia, and are not included in the analysis) are included in the propofol pharmacokinetic analysis data set.

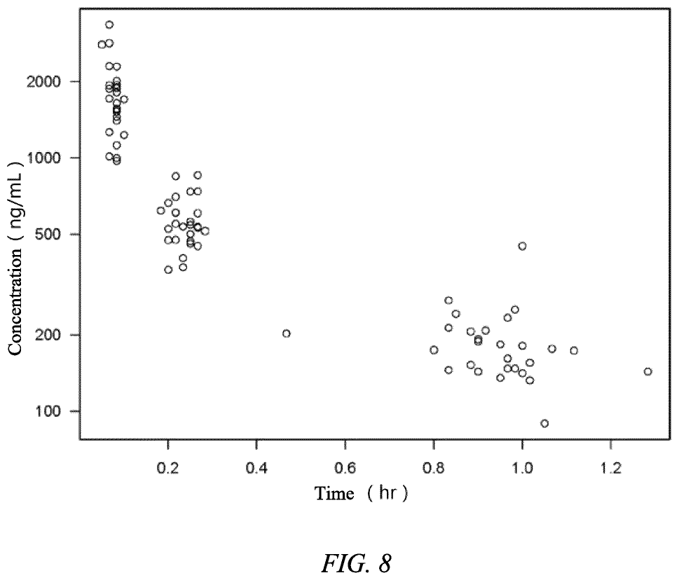

A pharmacokinetic data set included in the analysis are determined by evaluating the clinical trial data included:
plasma drug concentration data: acquired by means of implementation of the sampling scheme (see FIG. 8);
baseline demographic characteristic data: race, age, height, weight and gender;
blood biochemical index data: blood total protein content, creatinine clearance, glutamic-oxalacetic transaminase and glutamic-pyruvic transaminase; and
blood collection sites.

### 3. Processing of Data

Processing the data in step (2) comprising:
determining and processing the observations below the lower limit of detection comprising: determining the lower limit of detection by a detecting instrument, wherein the observations below the lower limit of detection are not used for population pharmacokinetic analysis, and if the proportion of the observations below the lower limit of detection is greater than 15%, investigating the influence of the observations below the lower limit of detection on model goodness-of-fit and modeling parameters by using a likelihood function method;
determining and processing the anomalous value data comprising: checking whether there are anomalous values in a sample according to a plasma concentration—time curve in subjects receiving drug administration, and eliminating the anomalous values;
determining and processing the outliers comprising: determining outliers according to residual analysis of preliminary modeling results, and eliminating the outliers; and
processing the missing covariates comprising: if the missing covariate for the subject is less than 15%, applying imputation with the median of the data set for continuous covariates, and applying imputation with a value of the most common category for categorical covariates; and if the missing covariate for the subject is greater than 15%, applying no imputation, and performing exploratory analysis on the PK parameters of the subject with full covariate information by using a Bayesian estimation method.

### 4. Establishment of a Preliminary Population Pharmacokinetic Foundation Model

Establishing the preliminary foundation model on the basis of the data processed in step 3 comprising:
(1) describing interindividual variation of the PK parameters by using the following equation:

$${θ}_{i} = exp ( {θ}_{T} + {η}_{i} ) ;$$

(2) describing variability of a residual by using the following equation:

$$log ( {y}_{ij} ) = log ( {\hat{y}}_{ij} ) + {ε}_{ij} ;$$

(3) comparing a one-compartment model, a two-compartment model and a three-compartment model by comparing whether the decrease of the objective function values of the two nested models is significant on the basis of the plasma drug concentration-time curve, and selecting the three-compartment model as shown in FIG. 1 as the preliminary foundation model, wherein CL represents central compartment clearance; V1 represents volume of distribution in the central compartment; V2 represents volume of distribution in the peripheral compartment 1; V3 represents volume of distribution in the peripheral compartment 2; Q2 represents intercompartmental clearance between the peripheral compartment 1 and the central compartment; Q3 represents intercompartmental clearance between the peripheral compartment 2 and the central compartment; RO represents infusion rate; and K represents elimination rate constant.

### 5. Establishment of a Final Population Pharmacokinetic Foundation Model

Establishing the final foundation model on the basis of the preliminary foundation model in step 4 comprising:
(1) determining covariates included in the evaluation on the basis of clinical knowledge and a drug action mechanism:
baseline demographic characteristic covariates: baseline values for age, gender, weight and race;
blood biochemical index covariates: creatinine clearance, total protein, glutamic-oxalacetic transaminase, glutamic-pyruvic transaminase, alkaline phosphatase and total bilirubin; and
blood collection sites.
(2) establishing a final population pharmacokinetic foundation model on the basis of the covariates included in the evaluation.

### 6. Establishment of the Population Pharmacokinetic Model

Establishing the population pharmacokinetic model by adding the screened covariates on the basis of the final foundation model in step 5 comprising:
(1) Pre-screening of covariates:
analyzing the correlation between the continuous covariates and the PK parameters by using the following equation:

$${θ}_{i} = {θ}_{pop} \cdot {( \frac{{Cov}_{i}}{{Cov}_{pop}} )}^{{k}_{co v}} ;$$

and analyzing the correlation between the categorical covariates and the PK parameters by using the following equation:

$${θ}_{i} = {θ}_{pop} \times exp ( {k}_{c ov} \cdot {X}_{i} )$$

(2) Final screening of the covariates by using a forward method and a backward method, and establishing the population pharmacokinetic model:
The forward method: sequentially adding each covariate to a preliminary structural model of the final foundation model in step (5), wherein with a log likelihood ratio test, if an objective function value is decreased by more than 6.63 after adding 1 covariate, the newly added covariate is considered significant, with p<0.01; and firstly adding the covariate having the most significant influence on the basis of the preliminary structural model to form an improved model, then testing the statistically significant covariate screened in the previous step with the improved model, and repeating the process until no significant covariates can be found to obtain a full model;
The backward method: a process of deleting the covariates one by one on the basis of the full model, wherein if an objective function value is increased by more than 10.83 after deleting 1 covariate, the deleted covariate is considered significant, with p<0.001.

A stepwise forward addition method is used for the propofol PopPK model in NONMEM. The results show that WT has a significant influence (p<0.01) on CL. The influence of WT on the CL of the propofol is eliminated by using a stepwise backward elimination method (Δ_{-2LL}=8.691).

In covariate screening, it is not found that covariates have a significant influence on the pharmacokinetic PK parameters of propofol.

### 7. Evaluation of the Population Pharmacokinetic Model

Evaluating the pharmacokinetic model established in step 6 comprising:
(1) Model goodness-of-fit (GOF) diagnostic plot

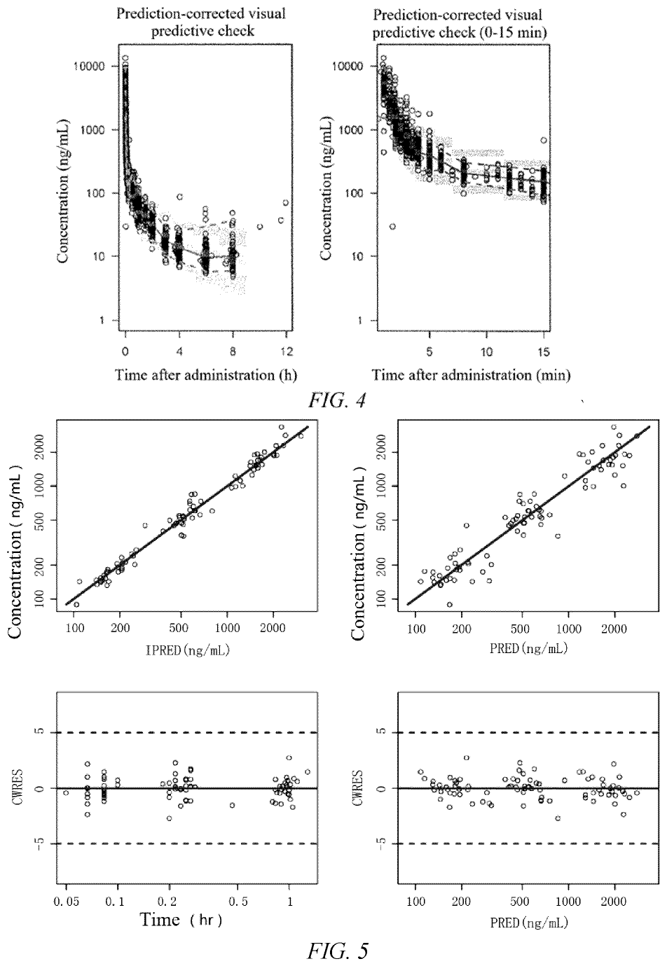

The diagnostic plots of the final PopPK model for the drug are as shown in FIG. 5. The results show that there is good consistency between an observed concentration value and a predicted concentration, and no significant deviations are observed in the plots of conditional weighted residuals versus time and versus a predicted concentration. In FIG. 5, the upper left panel is a diagnostic plot of observations versus individual predictions in the final model of the drug, the upper right panel is a diagnostic plot of observations versus population predictions in the final model of the drug, the lower left panel is a diagnostic plot of conditional weight residuals (CWRES) versus time, and the lower right panel is a diagnostic plot of CWRES versus population predictions, wherein solid lines in the middle are standard lines of 0, and dotted lines are auxiliary lines of |CWRES|=5.
(2) Prediction-corrected visual predictive check (pcVPC)

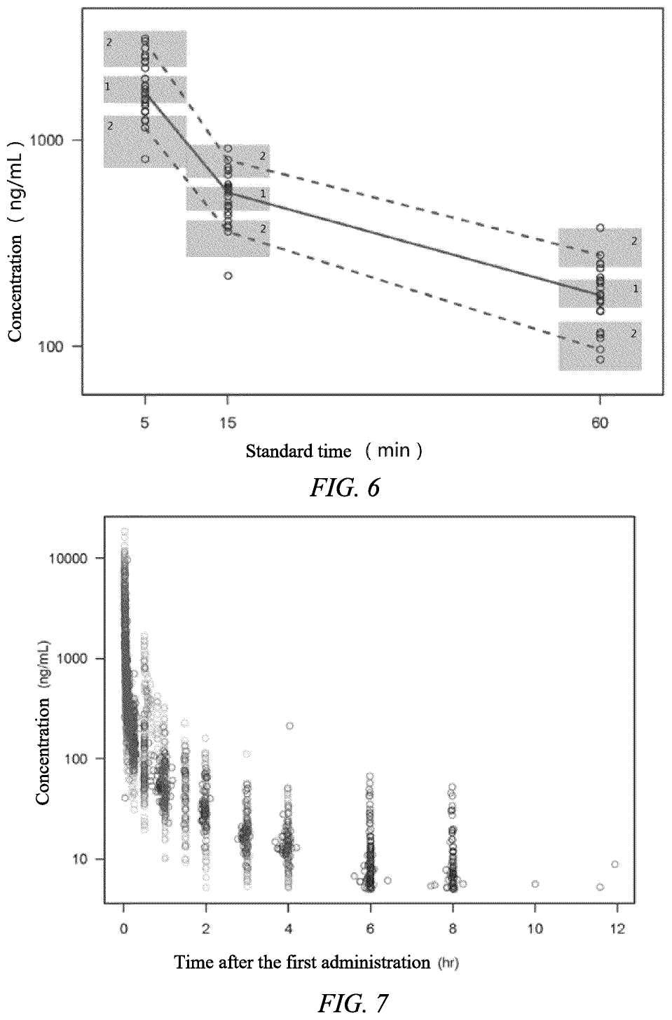

pcVPC is used to evaluate the ability of the model to reproduce the data distribution. 1000 trials are simulated and reproduced according to the observed covariate information of each subject, the estimated values of the final population pharmacokinetic model parameters, the random effects and the residual errors. The pc VPC of the plasma drug concentration-time curve is as shown in FIG. 6. FIG. 6 shows the observed concentrations (dots) of all subjects, the model-predicted 95% confidence (middle shaded area) interval for the median and the model-predicted 95% confidence intervals (upper and lower shaded areas) for the 2.5th and 97.5th percentiles. The results show that the final population pharmacokinetic model can fully predict the central tendency and variability of drug concentrations of all subjects in the clinical study. In FIG. 6, the circles are observations, the middle solid lines are medians of the observations, the upper and lower dashed lines correspond to the 97.5th and 2.5th percentiles respectively, the middle shaded area is the model-predicted 95% confidence interval for the median, the upper and lower shaded areas are 95% confidence intervals for the median lines and the 2.5th and 97.5th percentile lines for data of the 1000 simulations, and data lower than the lower limit of detection are not shown in this figure.
(3) Bootstrap

See Table 5 below for the comparison between the parameters estimated by the final model and the parameters estimated by Bootstrap. The median of the parameters estimated by Bootstrap is similar to the median of the parameters estimated by the final PopPK model. 95% CI of the parameters estimated by the final PopPK model is highly overlapped with 95% CI of the parameters estimated by Bootstrap (represented by the 2.5th to 97.5th percentile interval), indicating that the final model has relatively good stability and accuracy.

TABLE 5
Parameters estimated by final model and parameters estimated by Bootstrap

| | | Typical values of | Median of Bootstrap |
|---|---|---|---|
| | | population model | population model parameters |
| Parameters | Parameter description | parameters (95% CI) | (2.5^{th} to 97.5^{th} percentiles) |
| exp(θ₁) | Clearance, CL (L/hr) | 95.2 (76.6 to 114) | 91.6 (25.1 to 114) |
| exp(θ₂) | Volume of distribution in the | 9.46 (4.83 to 14.1) | 8.92 (1.15 to 17.2) |
| | central compartment, V₁ (L) | | |
| exp(θ₃) | Intercompartmental clearance | 142 (88.4 to 196) | 132 (13.0 to 200) |
| | between the central | | |
| | compartment and the | | |
| | peripheral compartment 1, Q₂ | | |
| | (L/hr) | | |
| exp(θ₄) | Volume of distribution in the | 37.6 (25.2 to 50.0) | 35.6 (1.64 to 53.3) |
| | peripheral compartment 1, V₂ | | |
| | (L) | | |
| exp(θ₅) | Clearance between the central | 48.6, FIX(—) | 48.6, FIX(—) |
| | compartment and the | | |
| | peripheral compartment 2, Q₃ | | |
| | (L/hr) | | |
| exp(₆) | Volume of distribution in the | 262, FIX(—) | 262, FIX(—) |
| | peripheral compartment 2, V₃ | | |
| | (L) | | |
| ω_{CL} | Clearance, CL % | 23.0 (14.8 to 31.1) | 22.5 (13.6 to 30.6) |
| ω_{V1} | Volume of distribution in the | 30.5 (8.11 to 52.9) | 32.4 (6.02 to 49.5) |
| | central compartment, V₁% | | |
| σ₁ | Residual (%) | 19.2 (12.7 to 25.7) | 17.1 (11 to 23.1) |

(4) Shrinkage

The shrinkage values of the final model parameters of propofol are as shown in Table 6. The circles are observations, the solid lines are medians of the observations, the upper and lower dashed lines correspond to the 97.5th and 2.5th percentiles respectively, the shaded area marked as 1 is the model-predicted 95% confidence interval for the median, the shaded areas marked as 2 are 95% confidence intervals for the median lines and the 2.5th and 97.5th percentile lines for data of the 1000 simulations, and data lower than the lower limit of detection are not shown in this figure. In the final population pharmacokinetic model of propofol, the shrinkage amplitude of ω_{V1} is slightly higher than 30%.

TABLE 6
Shrinkage values of final model parameters of propofol

| | | Shrinkage |
|---|---|---|
| Parameters | Parameter description | values (%) |
| ωCL | Clearance, CL % | 13.2% |
| ωV1 | Volume of distribution in | 31.3% |
| | the central compartment, V1% | |
| σ1 | Residual (%) | 22.8% |

### Conclusion

The three-compartment linear pharmacokinetic model can well describe the pharmacokinetic characteristics of propofol in the dosage range in this study. After model evaluation, it shows that the final model has relatively good stability, accuracy and good prediction performance.

It is not found that covariates have a significant influence on the pharmacokinetic parameters of propofol.

### EXAMPLE 3

This example provides a system for determining clinical individual administration parameters of a compound of formula (I).

The system comprises a data acquisition device, a data processing device and a result output device.

The data comprise baseline demographic characteristic data, blood biochemical index data and blood collection sites. With the data processing device, individual administration parameter (dosage) results of the compound of formula (I) are obtained by using the following equation:

$${CL}_{i} = exp ( 4.2 + 0.349 ) \cdot log ( WT / 63.9 ) - 0.749 \cdot log ( TP / 72.4 ) + 0.238 \cdot SITE + {η}_{CL , i} ) ;$$

$${V}_{1 i} = exp ( 0.908 + 0.426 \cdot log ( AGE / 27 ) + {η}_{{V}_{1} , i} )$$

$${Q}_{2 i} = exp ( 4.06 + {η}_{{Q}_{2} , i} )$$

$${V}_{2 i} = exp ( 1.75 )$$

$${Q}_{3 i} = exp ( 4.08 + {η}_{{Q}_{3} , i} )$$

$${V}_{3 i} = exp ( 4.36 + {η}_{{V}_{3} , i} )$$

wherein CLᵢ represents central compartment clearance of the ith subject; when collecting a sample from venous blood, SITE=0, and when collecting a sample from artery blood, SITE=1; WT represents weight; TP represents total protein; η_{CL,i} is interindividual variation of the CL of the ith subject; and η follows a normal distribution with mean 0 and variance ω2, wherein the ω2 is a diagonal element of a variance-covariance matrix Ω of the interindividual variation;
wherein V₁ᵢ represents volume of distribution in the central compartment of the ith subject;
V₂ᵢ represents volume of distribution in the peripheral compartment 1 of the ith subject;
V₃ᵢ represents volume of distribution in the peripheral compartment 2 of the ith subject;
Q₂ᵢ represents intercompartmental clearance between the peripheral compartment 1 and the central compartment of the ith subject;
Q₃ᵢ represents intercompartmental clearance between the peripheral compartment 2 and the central compartment of the ith subject;
AGE represents age; and η represents interindividual variation of a corresponding parameter.

### Example 4

This example provides a system of individual administration parameters of propofol. The system comprises a data acquisition device, a data processing device and a result output device. With the data processing device, the individual administration parameter (dosage) results of propofol are obtained by using the following equation:

$${CL}_{i} = exp ( 4.56 + {η}_{CL , i} )$$

$${V}_{1 i} = exp ( 2.25 + {η}_{{V}_{1} , i} )$$

$${Q}_{2 i} = exp ( 4.96 )$$

$${V}_{2 i} = exp ( 3.63 )$$

$${Q}_{3 i} = exp ( 3.88 )$$

$${V}_{3 i} = exp ( 5.57 )$$

wherein CLᵢ represents central compartment clearance of the ith subject; η_{CL,i} is interindividual variation of the CL of the ith subject; and η follows a normal distribution with mean 0 and variance ω2, wherein the ω2 is a diagonal element of a variance-covariance matrix Ω of the interindividual variation;
wherein V₁ᵢ represents volume of distribution in the central compartment of the ith subject;
V₂ᵢ represents volume of distribution in the peripheral compartment 1 of the ith subject;
V₃ᵢ represents volume of distribution in the peripheral compartment 2 of the ith subject;
Q₂ᵢ represents intercompartmental clearance between the peripheral compartment 1 and the central compartment of the ith subject;
Q₃ᵢ represents intercompartmental clearance between the peripheral compartment 2 and the central compartment of the ith subject;
AGE represents age; and η represents interindividual variation of a corresponding parameter.

What is claimed is:

1. A method for inducing and maintaining anesthesia, comprising administering the compound of formula (I) or propofol to a subject, wherein, dosage regimen of the compound of formula (I) or propofol is derived from a population pharmacokinetic model, obtained through the following methods: 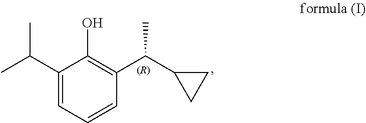
wherein an equation of pharmacokinetic parameters in the population pharmacokinetic model of the compound of formula (I) comprises:
$${CL}_{i} = exp ( 4.2 + 0.349 ) \cdot log ( WT / 63.9 ) - 0.749 \cdot log ( TP / 72.4 ) + 0.238 \cdot SITE + {η}_{CL , i} ) ;$$
an equation of pharmacokinetic parameters in the population pharmacokinetic model of propofol comprises:
$${CL}_{i} = exp ( 4.56 + {η}_{CL , i} )$$
wherein CLᵢ represents central compartment clearance of the ith subject; when collecting a sample from venous blood, SITE=0, and when collecting a sample from artery blood, SITE=1; WT represents weight; TP represents total protein; η_{CL,i} is interindividual variation of the CL of the ith subject; and η follows a normal distribution with mean 0 and variance ω2, wherein the ω2 is a diagonal element of a variance-covariance matrix Ω of the interindividual variation;
wherein the method comprises the following steps:
(1) acquisition of data;
(2) determination of data included in an analysis;
(3) processing of data;
(4) establishment of a preliminary population pharmacokinetic foundation model;
(5) establishment of a final population pharmacokinetic foundation model;
(6) establishment of the population pharmacokinetic model; and
(7) evaluation of the population pharmacokinetic model;
wherein the data in step (1) are derived from clinical trial data;
wherein step (2) comprises determining a pharmacokinetic data set included in the analysis by evaluating the clinical trial data included; the clinical trial data included comprise plasma drug concentration data, baseline demographic characteristic data, blood biochemical index data and blood collection sites; the baseline demographic characteristic data comprise any combination of two or more of race, age, height, weight and gender; and the blood biochemical index data comprise any combination of two or more of blood total protein content, creatinine clearance, glutamic-oxalacetic transaminase, glutamic-pyruvic transaminase, alkaline phosphatase and total bilirubin;
wherein step (3) comprises determining and processing one or a combination of two or more of observations below a lower limit of detection, anomalous value data, outliers and missing covariates;
determining and processing the observations below the lower limit of detection comprises: determining the lower limit of detection by a detecting instrument, wherein the observations below the lower limit of detection are not used for population pharmacokinetic analysis, and if the proportion of the observations below the lower limit of detection is greater than 15%, investigating the influence of the observations below the lower limit of detection on model goodness-of-fit and modeling parameters by using a likelihood function method;
determining and processing the anomalous value data comprises: checking whether there are anomalous values in a sample according to a plasma concentration—time curve in subjects receiving drug administration, and eliminating the anomalous values;
determining and processing the outliers comprises: determining outliers according to residual analysis of preliminary modeling results, and eliminating the outliers; and
processing the missing covariates comprises: if the missing covariate for the subject is less than 15%, applying imputation with the median of the data set for continuous covariates, and applying imputation with a value of the most common category for categorical covariates; and if the missing covariate for the subject is greater than 15%, applying no imputation, and performing exploratory analysis on the PK parameters of the subject with full covariate information by using a Bayesian estimation method;
wherein, the population pharmacokinetic model of formula (I) and propofol established are used for guiding the dosage regimen in the clinical trials, comprising estimating individual PK parameters of a subject by using a Bayesian post-hoc method on the basis of the population pharmacokinetic model, and simulating a plasma drug concentration—time curve of the propofol derivative or propofol administered by intravenous infusion according to the actual dosages of administration so as to calculate the area under the plasma drug concentration-time curve in different time periods, giving the reasonable dosage regimen combine with the data analysis of correlation between the exposure and the drug efficacy and safety, and administering the compound of formula (I) or propofol to the subjects according to the dosage regimen;
wherein, the population pharmacokinetic model analysis software used is NONMEM 7.

2. The method according to claim 1, wherein
the equation of the pharmacokinetic parameters in the population pharmacokinetic model of the compound of formula (I) further comprises:
$${V}_{1 i} = exp ( 0.908 + 0.426 \cdot log ( AGE / 27 ) + {η}_{{V}_{1} , i} )$$
$${Q}_{2 i} = exp ( 4.06 + {η}_{{Q}_{2} , i} )$$
$${V}_{2 i} = exp ( 1.75 )$$
$${Q}_{3 i} = exp ( 4.08 + {η}_{{Q}_{3} , i} )$$
$${V}_{3 i} = exp ( 4.36 + {η}_{{V}_{3} , i} )$$
the equation of the pharmacokinetic parameters in the population pharmacokinetic model of propofol further comprises:
$${V}_{1 i} = exp ( 2.25 + {η}_{{V}_{1} , i} )$$
$${Q}_{2 i} = exp ( 4.96 )$$
$${V}_{2 i} = exp ( 3.63 )$$
$${Q}_{3 i} = exp ( 3.88 )$$
$${V}_{3 i} = exp ( 5.57 )$$
wherein V₁ᵢ represents volume of distribution in the central compartment of the ith subject;
V₂ᵢ represents volume of distribution in the peripheral compartment 1 of the ith subject;
V₃ᵢ represents volume of distribution in the peripheral compartment 2 of the ith subject;
Q₂ᵢ represents intercompartmental clearance between the peripheral compartment 1 and the central compartment of the ith subject;
Q₃ᵢ represents intercompartmental clearance between the peripheral compartment 2 and the central compartment of the ith subject;
AGE represents age; and η represents interindividual variation of a corresponding parameter.

3. The method according to claim 1, wherein the anomalous value data comprise:
1) repeated concentration records at the same time point, except for concentrations in the artery and the vein at the same time point;
2) a trough concentration greater than a corresponding peak concentration;
3) an administration time point after the peak concentration;
4) an administration time point before the trough concentration;
5) concentration records after the end of intravenous administration and before the peak concentration; and
6) unexplained sudden drop or sudden rise in concentrations.

4. The method according to claim 1, wherein step (4) comprises comparing various structural models on the basis of a plasma drug concentration-time curve, wherein the optimal model is selected as a preliminary structural model, to form a preliminary foundation model with a residual model.

5. The method according to claim 4, wherein the preliminary structural model is a three-compartment model with zero-order absorption and first-order linear elimination from the central compartment, and parameters of the preliminary structural model comprise: central compartment clearance, CL; volume of distribution in the central compartment, V1; volume of distribution in the peripheral compartment 1, V2; volume of distribution in the peripheral compartment 2, V3; intercompartmental clearance between the peripheral compartment 1 and the central compartment, Q2; intercompartmental clearance between the peripheral compartment 2 and the central compartment, Q3; infusion rate, RO; and elimination rate constant K.

6. The method according to claim 1, wherein with the preliminary foundation model, interindividual variation of PK parameters is described by using the following equation:
Θᵢ=exp(θ_{T}+ηᵢ)
wherein θi represents a PK parameter of the ith subject; θ_{T} represents a natural logarithm of a population typical value of the PK parameter; and ηᵢ represents interindividual variation, and is a random variable following a normal distribution with mean 0 and variance ω², wherein the ω² represents a diagonal element of a variance-covariance matrix of the interindividual variation.

7. The method according to claim 1, wherein with the preliminary foundation model, variability of a residual is described by using the following equation:
$$log ( {y}_{ij} ) = log ( {\hat{y}}_{ij} ) + {ε}_{ij}$$
wherein yᵢⱼ represents the jth observed concentration of the ith subject, ŷᵢⱼ represents the jth model-predicted concentration of the ith subject, and εᵢⱼ represents a proportional residual of the jth observed concentration of the ith subject, wherein the observed concentration and the predicted concentration are independent of each other and follow a normal distribution with mean 0 and variance σ2, respectively.

8. The method according to claim 1, wherein step (5) comprises determining covariates included in the evaluation on the basis of clinical knowledge and a drug action mechanism, and establishing a final population pharmacokinetic foundation model on the basis of the covariates included in the evaluation.

9. The method according to claim 8, wherein the covariates included in the evaluation comprise baseline demographic characteristic covariates, blood biochemical index covariates and blood collection sites.

10. The method according to claim 9, wherein the baseline demographic characteristic covariates comprise any combination of two or more of baseline values for age, gender, weight and race; and the blood biochemical index covariates comprise any combination of two or more of creatinine clearance, total protein, glutamic-oxalacetic transaminase, glutamic-pyruvic transaminase, alkaline phosphatase and total bilirubin.

11. The method according to claim 1, wherein step (6) comprises
a) pre-screening of covariates; and
b) final screening of the covariates by using a forward method and a backward method, and establishing the population pharmacokinetic model.

12. The method according to claim 11, wherein the pre-screening of the covariates comprises:
analyzing the correlation between the PK parameters and each covariate by a graphic method, using linear regression for continuous covariates and using an analysis of variance test for categorical covariates; evaluating the data set on the basis of the model; estimating the parameters of the subject with the final foundation model by using a Bayesian estimation method; and estimating the influence of the covariates on the PK parameters.

13. The method according to claim 12, wherein the pre-screening of the covariates comprises:
analyzing the correlation between the continuous covariates and the PK parameters by using the following equation:
$${θ}_{i} = {θ}_{pop} \cdot {( \frac{{Cov}_{i}}{{Cov}_{pop}} )}^{{k}_{co v}} ;$$
and analyzing the correlation between the categorical covariates and the PK parameters by using the following equation:
$${θ}_{i} = {θ}_{pop} \times exp ( {k}_{c ov} \cdot {X}_{i} )$$
wherein θᵢ represents a PK parameter of the ith subject; θₚₒₚ represents a test population typical value of the PK parameter; Covᵢ represents a continuous covariate of the ith subject; Covₚₒₚ represents a median of the continuous variables in a test population; Xᵢ represents a categorical variable index of the ith subject, wherein value 0 represents the category with the most common category of a covariate, while other integer values represent other categories respectively; and K_{cov} represents a coefficient describing the influence of the covariate.

14. The method according to claim 13, wherein the final screening of the covariates comprises:
establishing a full model by using a forward method on the basis of the final foundation model, and then establishing the population pharmacokinetic model by using a backward method on the basis of the full model, wherein
the forward method comprises: sequentially adding each covariate to a preliminary structural model of the final foundation model in step (5), wherein with a log likelihood ratio test, if an objective function value is decreased by more than 6.63 after adding 1 covariate, the newly added covariate is considered significant, with p<0.01; and firstly adding the covariate having the most significant influence on the basis of the preliminary structural model to form an improved model, then testing the statistically significant covariate screened in the previous step with the improved model, and repeating the process until no significant covariates can be found; and
the backward method comprises: a process of deleting the covariates one by one on the basis of the full model, wherein if an objective function value is increased by more than 10.83 after deleting 1 covariate, the deleted covariate is considered significant, with p<0.001.

15. The method according to claim 1, wherein step (7) comprises evaluating the population pharmacokinetic model by using one or a combination of two or more of the following methods: a model goodness-of-fit diagnostic plot, a visual predictive check, bootstrap and shrinkage.

16. The method according to claim 15, wherein
the model goodness-of-fit diagnostic plot comprises one or a combination of two or more of the following figures: a relation diagram of a population predicted concentration and an observed concentration, a relation diagram of an individual predicted concentration and an observed concentration, a relation diagram of a conditional weighted residual and a population predicted concentration, and a relation diagram of a conditional weighted residual and time after first administration;
the visual predictive check comprises comparative mapping of a predicted result and a measured value according to final model parameters, covariates and actual dosage of administration so as to evaluate whether the population pharmacokinetic model can well describe a plasma drug concentration-time curve of a propofol derivative;
the bootstrap comprises repeatedly fitting with population pharmacokinetic model until 1000 data sets for bootstrap replication, and randomly selecting subject data and covariates for replacement to achieve the replication; and
the shrinkage comprises estimating individual parameter values of a subject by using a Bayesian estimation method with the population pharmacokinetic model, and calculating interindividual variations and individual residuals from model predictions and observations.

17. The method according to claim 16, wherein the shrinkage comprises evaluating the interindividual variations and individual residuals of the pharmacokinetic parameters by using the following equation, and quantifying the individual parameter values and random error estimates:
$${η}_{shrinkage} = 1 - \frac{SD ( {\hat{η}}_{p h} )}{ω}$$
$${ε}_{shrinkage} = 1 - SD ( IWRES ) ;$$
wherein η_{shrinkage} is interindividual variation, ε_{shrinkage} is an individual residual, ω is interindividual variation degree of individual parameter values estimated by the population pharmacokinetic model, ηₚₕ is η value of the parameter for all individuals, IWRES is an individual weighted residual; and SD represents standard deviation.

18. The method according to claim 1, wherein step (7) further comprises estimating individual PK parameters of the subject by using a Bayesian post-hoc method, simulating a plasma drug concentration-time curve of intravenous infusion according to actual dosages of administration, and calculating the area under the plasma drug concentration-time curve from 0-1 min, the area under the plasma drug concentration-time curve from 0-2 min, the area under the plasma drug concentration-time curve from 0-4 min, the area under the plasma drug concentration-time curve from 0-10 min, the area under the plasma drug concentration-time curve from 0-24 h and the peak concentration.

19. A system for use in the method for determining a dosage regimen of the compound of formula (I) or propofol as claimed in claim 1, the system comprising a data acquisition device, a data processing device and a result output device, 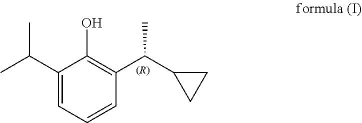
wherein the data comprise baseline demographic characteristic data, blood biochemical index data and blood collection site information data; with the data processing device, individual administration parameter results of the compound of formula (I) are obtained by using the following equation:
$${CL}_{i} = exp ( 4.2 + 0.349 ) \cdot log ( WT / 63.9 ) - 0.749 \cdot log ( TP / 72.4 ) + 0.238 \cdot SITE + {η}_{CL , i} ) ;$$
or individual administration parameter results of propofol are obtained by using the following equation:
$${CL}_{i} = exp ( 4.56 + {η}_{CL , i} )$$
wherein CLᵢ represents central compartment clearance of the ith subject; when collecting a sample from venous blood, SITE=0, and when collecting a sample from artery blood, SITE=1; WT represents weight; TP represents total protein; η_{CL,i} is interindividual variation of the CL of the ith subject; and η follows a normal distribution with mean 0 and variance ω2, wherein the ω2 is a diagonal element of a variance-covariance matrix Ω of the interindividual variation.

20. The system according to claim 19, wherein with the data processing device, individual administration parameter results of the compound of formula (I) are obtained by using the following equation:
$${CL}_{i} = exp ( 4.2 + 0.349 ) \cdot log ( WT / 63.9 ) - 0.749 \cdot log ( TP / 72.4 ) + 0.238 \cdot SITE + {η}_{CL , i} )$$
$${V}_{1 i} = exp ( 0.908 + 0.426 \cdot log ( AGE / 27 ) + {η}_{{V}_{1} , i} )$$
$${Q}_{2 i} = exp ( 4.06 + {η}_{{Q}_{2} , i} )$$
$${V}_{2 i} = exp ( 1.75 )$$
$${Q}_{3 i} = exp ( 4.08 + {η}_{{Q}_{3} , i} )$$
$${V}_{3 i} = exp ( 4.36 + {η}_{{V}_{3} , i} ) ;$$
or individual administration parameter results of propofol are obtained by using the following equation:
$${CL}_{i} = exp ( 4.56 + {η}_{CL , i} )$$
$${V}_{1 i} = exp ( 2.25 + {η}_{{V}_{1} , i} )$$
$${Q}_{2 i} = exp ( 4.96 )$$
$${V}_{2 i} = exp ( 3.63 )$$
$${Q}_{3 i} = exp ( 3.88 )$$
$${V}_{3 i} = exp ( 5.57 )$$
wherein V₁ᵢ represents volume of distribution in the central compartment of the ith subject;
V₂ᵢ represents volume of distribution in the peripheral compartment 1 of the ith subject;
V₃ᵢ represents volume of distribution in the peripheral compartment 2 of the ith subject;
Q₂ᵢ represents intercompartmental clearance between the peripheral compartment 1 and the central compartment of the ith subject;
Q₃ᵢ represents intercompartmental clearance between the peripheral compartment 2 and the central compartment of the ith subject;
AGE represents age; and η represents interindividual variation of a corresponding parameter.
